(19) United States
(12) Patent Application Publication
(10) Pub. No.: US 2007/0299405 A1
(43) Pub. Date: Dec. 27, 2007

(54) Structure and Method for Securing an Infusion Device

(76) Inventors: Heiner Kaufmann, Bern (CH); Florian Kuhni, Burgdorf (CH); Axel Remde, Lutzelfluh-Goldbach (CH); Gerald Studer, Sion (CH); Michael Haberli, Kussnacht (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/750,017

(22) Filed: May 17, 2007

(30) Foreign Application Priority Data
May 19, 2006 (CH) 816/06

(51) Int. Cl. A61M 25/02

(52) U.S. Cl. 604/180

(57) ABSTRACT

An infusion arrangement and method for administering a medicinal substance to a patient, the arrangement including a catheter head with an underside for securing to the patients' body, a cannula extending from the underside of the catheter head for insertion into the patient's body, a connector carried on the catheter head for establishing a releasable fluidic connection to an administration device, the administration device rotatably coupled to the connector, wherein the administration device carries, on peripheral areas of the underside thereof, at least one adhesive suitable for fixing the administration device in a selected position relative to the patient's body.

### CROSS-REFERENCE TO RELATED APPLICATION(S)

This application claims priority to Swiss Application No. 816/06, filed on May 19, 2006, which is hereby incorporated herein by reference in its entirety.

### BACKGROUND

The present invention relates to devices for dispensing, injecting, administering, infusing or delivering substances, and to methods of making and using such devices. More particularly, the present invention relates to securing an infusion device or a portion of an infusion device to a patient's skin, whereby the device may be reliably secured in a suitable position. The term “infusion device” is used herein interchangeably with and to refer to any device for dispensing, injecting, administering, infusing or delivering substances, including infusion pumps, administration devices, appliances, metering devices, etc.

Infusion devices, including miniaturized infusion devices, e.g., infusion pumps, that are applied directly to the surface of the skin and permit discrete and comfortable wearing are used, for example, for outpatient treatment of diabetes patients. In some instances, after such an infusion device has been secured to the surface of the skin, it generally adheres to the body for 4-5 days and may then be removed and reattached, or completely or partially disposed of. In many instances, the use of such infusion devices involves the initial application or use of an infusion set, catheter head or the like to the surface of the skin and the insertion of a cannula carried by the infusion set or catheter head into the body tissue. This procedure may be carried out by patients or care-givers.

In daily use by the patient, it is desirable that the infusion device, or at least part of it, can be temporarily removed, for example to allow the patient to freely pursue a sporting activity, or so as not to expose the precision parts or electronic components of a metering device, which may be associated with the infusion device, to undesired contact with water during bathing or showering.

To allow the infusion device to be temporarily removed without removing the cannula and thereafter requiring the cannula to be pushed through the skin again, infusion devices that may be detached from the catheter head are known. The catheter head and the cannula form an indwelling fixture and, being provided with suitable sealing means, remain in or on the body tissue during the disconnection.

Patent specification DE 199 12 459 C2 describes an indwelling fixture in the form of an adapter which can be applied to the surface of the skin by an adhesive skin plaster and which permits connection of a metering device for continuous administration of a medical liquid, or connection of an injection pen, or fluidic connection to a syringe needle.

If an infusion or metering device, e.g., a pump, is mechanically coupled to an indwelling fixture, the static imbalance of the infusion device, which cannot be entirely eliminated because of its construction, results in forces or tilting moments that are dependent on and/or variable with the orientation of the infusion device. Such forces may limit or effect the wearing comfort experienced by the person wearing the device. For a person wearing a device, such as an infusion pump, it is desirable to permit individual orientation of the pump such that it can be worn in a suitable, comfortable position on the body.

### SUMMARY

An object of the present invention is to provide that an infusion device coupled to an indwelling fixture via a releasable plug-in connection and fluidic connection can be affixed comfortably and in a suitable position to the surface of the skin and, after disconnection, can be safely and comfortably secured again.

An arrangement and method for administering a medicinal substance to a patient, the arrangement including a catheter head with an underside for securing to the patients' body, a cannula extending from the underside of the catheter head for insertion into the patient's body, a connector carried on the catheter head for establishing a releasable fluidic connection to an administration device, the administration device at least initially rotatably coupled to the connector, wherein the administration device carries, on peripheral areas of the underside thereof, at least one adhesive suitable for fixing the administration device in a selected position relative to the patient's body.

In one embodiment, the present invention comprises an infusion device which is worn on the surface of the skin and administers a medical liquid, the infusion device being releaseably connected to a catheter head rotatably about a guide element associated with or carried by the catheter head. The catheter head is secured to the surface of the skin by an adhesive, as is known in the prior art.

When the infusion device is connected on or to the catheter head, it initially remains free to rotate about the guide element. In this way, the person wearing the device is able to orient the device individually with respect to, for example, wearing comfort, discreetness, convenience of use, etc. As soon as the device is located in the desired position, the device can be fixed and secured by adhesives that are located on a peripheral area or areas of the underside. In some embodiments, a second securing area, which may be formed and/or located similarly, permits a double introduction of forces, either redundant or different forces, with an overall better fixing to the surface of the skin. The reliability can be further improved by using several adhesives, which may be similar or different.

To disconnect the infusion device temporarily, for example to protect the it from undesired contact with water and moisture during bathing or showering, the adhesive attachment in the peripheral area is first removed. The infusion device is then released from the catheter head.

To ensure a satisfactory adhesive connection or reconnection, new adhesives or adhesives with characteristics facilitating reuse may be used on the peripheral areas after a temporary disconnection.

In one embodiment, the present invention comprises an infusion device for administering a liquid medical substance into human or animal body tissue, said device comprising a catheter head with an underside for securing to the body tissue, a cannula which is mounted on the catheter head, protrudes from the underside thereof and is inserted into the body tissue, a guide element and connector arranged on the top face of the catheter head and designed to establish a releasable plug-in connection and fluidic connection to an administration device, the administration device mounted, at least initially, so as to be able to rotate about the guide element and establish a releasable connection to the catheter head, wherein the administration device is prepared, on the peripheral areas, with at least one adhesive suitable for fixing the administration device onto the body such that the latter can be suitably worn on the body.

In one preferred embodiment of the present invention, plaster-like adhesives are provided on the underside of an infusion device, the active adhesive surface covered initially by a protective film. When the plaster-like adhesive is to be activated, the protective cover is removed. An ergonomically designed tear-off tab on the protective film makes it easier for the person using the device to grip the film and pull it off.

In another embodiment, separately stored adhesives provided as auxiliaries are not activated until directly before use on the infusion device, and are applied at a suitable location on the underside of the device.

In another preferred embodiment, adhesives are secured on the underside of the device via one or more independent backing films. In this way, used adhesives can be removed as and when required.

### BRIEF DESCRIPTION OF THE DRAWINGS

FIG. 1 is a side view of one embodiment of a catheter head in accordance with the present invention.

FIG. 2 depicts the underside of one embodiment of an infusion device in accordance with the present invention.

### DETAILED DESCRIPTION

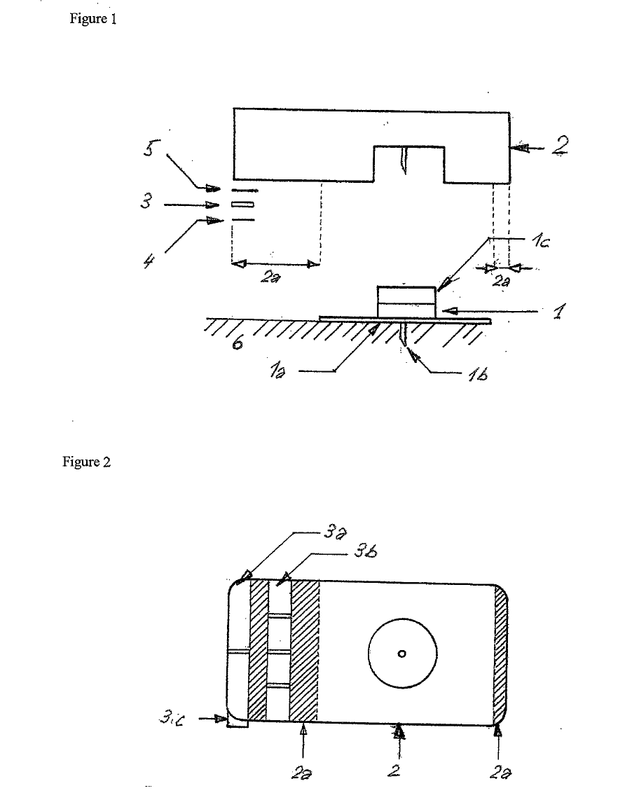

FIG. 1 shows a side view of a catheter head 1 which is secured to the surface of the skin by an adhesive or adhesives 1a applied on the underside thereof. A cannula lb extends from the underside to penetrate into the body tissue 6. An administration or infusion device in the form of an insulin pump is pushed on over the guide element 1c and then connected fluidically and mechanically. The insulin pump 2 remains rotatable and is free to turn about the guide element 1c, which allows the person wearing the pump to orient the pump suitably for comfortable application to the body. Only after the person wearing the pump has fixed it in place by the use of adhesive 1a is the pump fully secured on the body. Reliable fastening is achieved through the additionally obtained securing and/or the introduction of forces.

The arrangement of a suitable protective film 4, a suitable adhesive in the form of a skin-compatible plaster 3, and a suitable backing film 5 can be seen exploded at the left side of FIG. 1. Each of these layers can be selected from suitable or preferred material, depending, for example, on the intended use, patient characteristics, selected adhesive, etc.

FIG. 2 shows the underside of an infusion device with different adhesive arrangements located in the peripheral areas 2a. Adhesive arrangement 3a consists of an adhesive plaster which is suitable for small gravitational forces and small acceleration forces. Adhesive arrangement 3b comprises several adhesives which are able to take up greater forces and acceleration components than in adhesive arrangement 3a. To make the protective film 4 or backing film 5 easier to access, a gripping tab 3c is formed by the film contour. The adhesive arrangements mentioned can be present several times, e.g., in successive layers, on the peripheral areas 2a, such that an unused adhesive is available for each renewed connecting procedure.

Embodiments of the present invention, including preferred embodiments, have been presented for the purpose of illustration and description. They are not intended to be exhaustive or to limit the invention to the precise forms and steps disclosed. Modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An infusion device for administering a medical liquid into human or animal body tissue, said device comprising:
a) a catheter head with an underside for securing to the body tissue;
b) a cannula mounted on the catheter head, protruding from the underside thereof for insertion into the body tissue;
c) a guide element and connector arranged on a top face of the catheter head to establish a releasable plug-in connection and fluidic connection to an administration device;
d) the administration device releaseably mounted to the catheter head so as to be able to rotate about the guide element; wherein
e) the administration device carries, on a peripheral area, securing means for affixing the administration device to the body.

2. The infusion device according to claim 1, wherein the securing means comprises a pressure-sensitive skin-compatible adhesive or plaster.

3. The infusion device according to claim 2, wherein a protective film with at least one tear-off tab overlies the adhesive.

4. The infusion device according to claim 3, wherein the securing means comprises several adhesives, and the several adhesives are covered by a common protective film.

5. The infusion device according to claim 4, wherein the several adhesives include at least two adhesives having different characteristics with respect to each other.

6. The infusion device according to claim 1, wherein the securing means comprises one or more adhesives connected to a portion of an underside of the administration device via a removable backing film.

7. An infusion arrangement for administering a medical liquid into human or animal body tissue, said arrangement comprising:
a catheter head with an underside adapted for securing the catheter head to the body tissue;
a cannula carried by the catheter head and extending from the underside thereof for insertion into the body tissue;
a connector carried on a top face of the catheter head for establishing a releasable fluidic connection to an administration device, wherein, when the administration device is connected to the connector, the administration device is able to rotate about the connector, and wherein the administration device carries at least one adhesive for affixing the administration device to the body.

8. The infusion arrangement according to claim 7, wherein the administration device is rotatable relative to the connector when initial connected thereto and later may be fixed relative to the connector.

9. The infusion arrangement according to claim 8, wherein the at least one adhesive is overlaid by a removeable film.

10. The infusion arrangement according to claim 9, wherein the adhesive is biocompatible.

11. The infusion arrangement according to claim 10, wherein the at least one adhesive is on an underside of the administration device.

12. The infusion arrangement according to claim 11, wherein the at least one adhesive is in at least a peripheral area of the underside.

13. The infusion arrangement according to claim 7, wherein the at least one adhesive comprises at least two adhesives having different characteristics with respect to each other.

14. The infusion arrangement according to claim 13, wherein the at least two adhesives are overlaid by a removeable film.
